(19) United States
(12) Patent Application Publication
(10) Pub. No.: US 2025/0010018 A1
(43) Pub. Date: Jan. 9, 2025

(54) SYSTEMS AND METHODS FOR USING OLFACTORY STIMULI TO INFLUENCE A BEHAVIOR OR CONDITION OF A SUBJECT

(71) Applicants: University of Pittsburgh-Of the Commonwealth System of Higher Education, Pittsburgh, PA (US); The Board of Regents of the University of Oklahoma, Norman, OK (US)

(72) Inventors: Michael A. Sayette, Pittsburgh, PA (US); Rachel S. Herz, Warwick, RI (US); Michael Businelle, Norman, OK (US)

(21) Appl. No.: 18/765,731

(22) Filed: Jul. 8, 2024

Related U.S. Application Data
(60) Provisional application No. 63/512,169, filed on Jul. 6, 2023.

(51) Int. Cl. A61M 21/00

(52) U.S. Cl. CPC A61M 21/00; A61M 2021/0016; A61M 2205/3306; A61M 2205/3389; A61M 2205/3592; A61M 2205/50

(57) ABSTRACT

A system may include a plurality of compartments, wherein each compartment defines a respective internal volume. A system may include a plurality of odorous materials, wherein each compartment houses a respective odorous material within the respective internal volume. A system may include an integrated circuit (IC) chip comprising a processor and a memory, the memory having computer-executable instructions stored thereon that, when executed by the processor, cause the IC chip to monitor a respective state of each compartment.

### CROSS-REFERENCE TO RELATED APPLICATIONS

This application claims the benefit of U.S. provisional patent application No. 63/512,169, filed on Jul. 6, 2023, and titled “SYSTEMS AND METHODS FOR USING OLFACTORY STIMULI TO INFLUENCE A BEHAVIOR OR CONDITION OF A SUBJECT,” the disclosure of which is expressly incorporated herein by reference in its entirety.

### STATEMENT REGARDING FEDERALLY FUNDED RESEARCH

This invention was made with government support under Grant nos. AT010896 and CA10605 awarded by the National Institutes of Health. The government has certain rights in the invention.

### BACKGROUND

Many people have urges to engage in unhealthy behaviors (e.g., smoking, overeating) that they want to avoid doing. In some cases, they wish to refrain from these actions permanently, while in other instances it may be for a discrete period of time. A number of approaches have been considered. Pharmacologic approaches, such as psychotropic medications (e.g., Bupropion), or nicotine gum, have reduced cravings for some people. Yet for many, these methods fall short and have disadvantages, such as generating aversive side effects, being addictive in their own right, and being slow acting. Nonpharmacological (behavioral) approaches to curbing cravings also have been employed. These include meditation, coping skills, and exercise. While these behavioral methods have shown some positive effects, for many people they also have their drawbacks, including being inconvenient to use and hard to master. There is a clear need for novel approaches to craving reduction.

Recently, methods based on olfactory science have emerged as worthy areas of inquiry. It has long been understood that olfactory cues (OCs) can cause cravings (e.g., the scent of freshly baked bread can elicit hunger). Less appreciated is the idea that OCs can serve as a craving reduction tool. Cravings are emotional states, and emotions are better manipulated through olfaction than other senses. Research indicates a strong overlap of neural circuitry between olfaction and emotion. Both OCs and craving draw upon limited capacity attentional resources. Thus, OCs may compete with cravings for the same pool of attentional resources, and therefore may be especially well suited for relieving cravings for a variety of behaviors.

### SUMMARY

Systems and methods for using olfactory stimuli to influence behavior or condition of a subject are described herein. The systems and methods offer a comprehensive solution for controlling cravings and thereby remediating a wide variety of behaviors, including but not limited to smoking, alcohol consumption, other drug use, food intake, and a range of compulsive behaviors. The systems and methods allow the subject to have voluntary control to smell odors either when prompted or when self-initiated. While there have been some efforts to develop methods for relieving cravings and stress, many people still struggle to regulate their actions, due in part to overwhelming cravings. Thus, the systems and methods can be used to curb urges in the behavior of concern (e.g., smoking) and reduce stress, irrespective of whether the individual's ultimate goal is to permanently terminate the behavior (e.g., quit smoking). Additionally, in situations when the behavior is frowned upon or prohibited, such as when the subject is on an airplane, the systems and methods may help the subject cope with and/or attenuate urges.

In some aspects, the techniques described herein relate to a system for using olfactory stimuli to influence a behavior or condition of a subject including: a container including: a plurality of compartments, wherein each compartment defines a respective internal volume; and a plurality of odorous materials, wherein each compartment houses a respective odorous material within the respective internal volume; and an integrated circuit (IC) chip including a processor and a memory, the memory having computer-executable instructions stored thereon that, when executed by the processor, cause the IC chip to monitor a respective state of each compartment.

In some aspects, the respective state of each compartment is at least one of an opened state, a closed state, a locked state, or an unlocked state.

In some aspects, the memory has further computer-executable instructions stored thereon that, when executed by the processor, cause the IC chip to record the respective state of each compartment.

In some aspects, the memory has further computer-executable instructions stored thereon that, when executed by the processor, cause the IC chip to transmit the respective state of each compartment to a remote computing device.

In some aspects, the memory has further computer-executable instructions stored thereon that, when executed by the processor, cause the IC chip to generate a control signal configured to prompt the subject to open a specific compartment of the container.

In some aspects, each odorous material includes a liquid, an aerosolized liquid, a solid, a polyurethane, a plastic, a powder, a gel, a nanoemulsion, a wax, a paper, a celluloid material, a gum base, a foam, or a composite.

In some aspects, each of the odorous materials is configured to emit a distinct odor. Optionally, the distinct odor is a lemon odor, a vanilla odor, a floral odor, a peppermint odor, an apple odor, or a chocolate odor.

In some aspects, each compartment further includes a respective cover configured to restrict access to the respective internal volume. Optionally, each compartment further includes a respective lock.

In some aspects, each compartment further includes a respective seal configured to isolate the respective internal volume from an external environment.

In some aspects, each compartment further includes a respective marking to indicate the respective odorous material within the respective internal volume.

In some aspects, the system further includes a remote computing device. The IC chip and the remote computing device are operably coupled by a communication link. Optionally, the communication link is a wireless communication link.

In some aspects, the remote computing device is configured to receive a signal indicating the respective state of each compartment.

In some aspects, the remote computing device is configured to measure a respective amount of time each compartment is in an opened state.

In some aspects, the remote computing device is configured to: receive an image of the container; and analyze the image to determine the respective state of each compartment.

In some aspects, the remote computing device is configured to prompt the subject to smell an odor contained in a specific compartment of the container.

In some aspects, the remote computing device is configured to receive subject-reported information about the odor contained in the specific compartment of the container.

In some aspects, the remote computing device is configured to: receive, from a wearable sensor, a physiological signal associated with the subject; and analyze the physiological signal, wherein the subject is prompted to smell the odor contained in the specific compartment of the container based, at least in part, on the analysis.

In some aspects, the remote computing device is configured to receive, from a location sensor, a location of the subject, wherein the subject is prompted to smell the odor contained in the specific compartment of the container based, at least in part, on the location of the subject.

In some aspects, the subject is prompted to smell the odor contained in the specific compartment of the container at a specific time.

In some aspects, the container is portable.

In some aspects, the techniques described herein relate to a method including: providing a container configured to release a plurality of odors to a subject; and prompting the subject to smell at least one of the odors released from the container, wherein one or more olfactory stimuli associated with the at least one of the odors influences a behavior or condition of the subject.

In some aspects, the method further includes treating the behavior or condition of the subject using the one or more olfactory stimuli.

In some aspects, the behavior or condition is smoking, alcohol consumption, drug use, food intake, stress, memory, an emotional state, a compulsion, or an unwanted behavioral or mental state.

In some aspects, the method further includes prompting the subject to release the at least one of the odors from the container.

In some aspects, the container includes: a plurality of compartments, wherein each compartment defines a respective internal volume; and a plurality of odorous materials, wherein each compartment houses a respective odorous material within the respective internal volume.

In some aspects, the container is portable.

In some aspects, the method further includes: receiving a signal indicating a respective state of each compartment; and measuring a respective amount of time each compartment is in an opened state.

In some aspects, the method further includes: receiving an image of the container; and analyzing the image to determine a respective state of each compartment.

In some aspects, the method further includes receiving subject-reported information about the at least one of the odors released from the container.

In some aspects, the method further includes: receiving a physiological signal associated with the subject; and analyzing the physiological signal, wherein the subject is prompted to smell the at least one of the odors released from the container based, at least in part, on the analysis.

In some aspects, the method further includes receiving a location of the subject, wherein the subject is prompted to smell the at least one of the odors released from the container based, at least in part, on the location of the subject.

In some aspects, each of the odors is a distinct odor. Optionally, the distinct odor is a lemon odor, a vanilla odor, a floral odor, a peppermint odor, an apple odor, or a chocolate odor.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a block diagram of an example system using olfactory stimuli to influence a behavior or condition of a subject according to an implementation described herein.

FIG. 2A illustrates an example container according to an implementation described herein. FIG. 2B illustrates a compartment of the container shown in FIG. 2A.

FIG. 3 is a flowchart illustrating example operations for using olfactory stimuli to influence a behavior or condition of a subject according to an implementation described herein.

FIG. 4 is an example computing device.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. As used herein, the terms “about” or “approximately” when referring to a measurable value such as an amount, a percentage, and the like, is meant to encompass variations of +20%, +10%, +5%, or +1% from the measurable value.

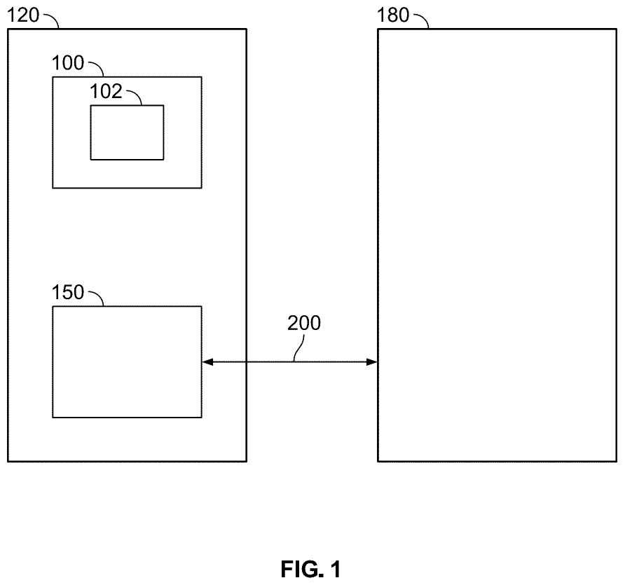

Referring now to FIG. 1, a block diagram of a system 120 for using olfactory stimuli to influence a behavior or condition of a subject is described. The term “subject” is defined herein to include animals such as mammals, including, but not limited to, primates (e.g., humans), cows, sheep, goats, horses, dogs, cats, rabbits, rats, mice and the like. In some implementations, the subject is a human. Additionally, as described herein, the behavior or condition can be smoking, alcohol consumption, drug use, food intake, stress, memory, an emotional state, or a compulsion. It should be understood that the above behaviors and conditions are provided only as examples. This disclosure contemplates that the behavior or condition may be another behavior or condition including, but not limited to, an unwanted behavioral or mental state.

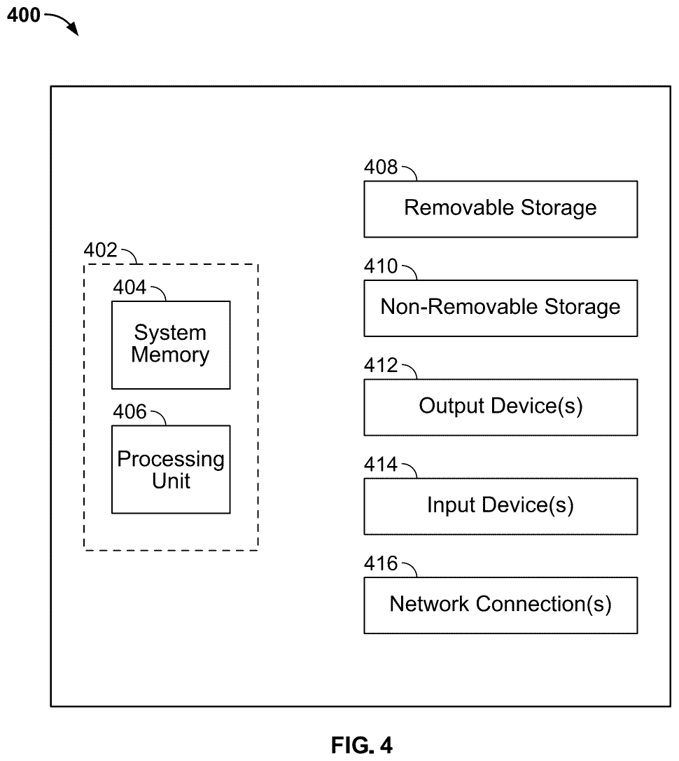

The system 120 can include a container 100 and an integrated circuit (IC) chip 150. Optionally, the container 100 is portable. Additionally, the container 100 can include a plurality of compartments 102. In some implementations, the IC chip 150 is integrated into the container 100. In other implementations, the IC chip 150 is attached to the container 100. In yet other implementations, the IC chip 150 is separate from the container 100. The IC chip 150 can include at least one processor and at least one memory operably coupled to the at least one processor. A basic configuration for an IC chip is illustrated in FIG. 4 by dashed line 402. The IC chip 150 can be programmed to perform one or more logical operations described in further detail below. Optionally, in some implementations, the system 120 can include a remote computing device 180. The remote computing device 180 can optionally be a portable computing device such as a smartphone, smartwatch, tablet, or laptop computer. It should be understood that a smartphone, smartwatch, tablet, and laptop computer are only provided as example portable computing devices. This disclosure contemplates that remote computing device 180 can be another portable computing device or non-portable computing device. An example computing device 400 is illustrated in FIG. 4. This disclosure contemplates that the remote computing device 180 can be programmed to perform one or more logical operations described in further detail below.

Additionally, the IC chip 150 and the remote computing device 180 can be operably coupled by a communication link 200. This disclosure contemplates the communication link 200 is any suitable communication link. For example, a communication link may be implemented by any medium that facilitates data exchange between the IC chip 150 and the remote computing device 180 including, but not limited to, wired, wireless and optical links. Optionally, the communication link 200 is a wireless communication link. In some implementations, the wireless communication link is a low power wireless communication link such as Bluetooth.

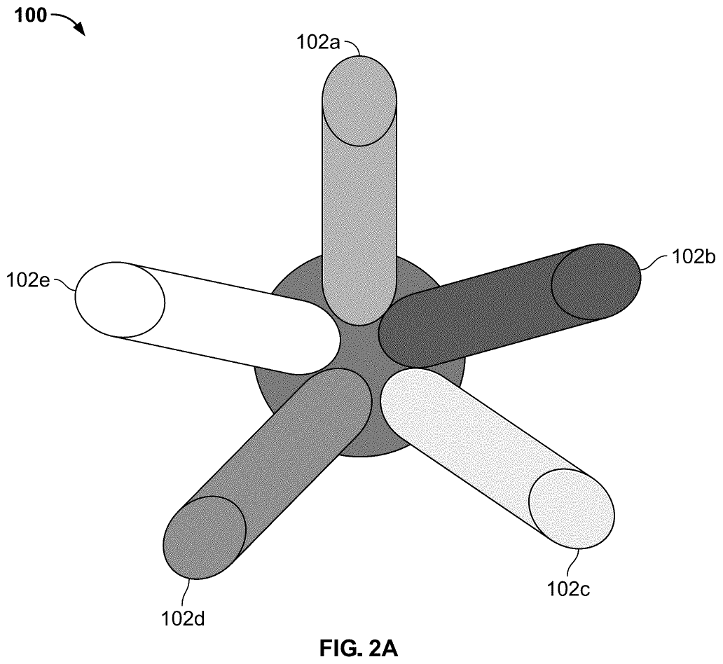

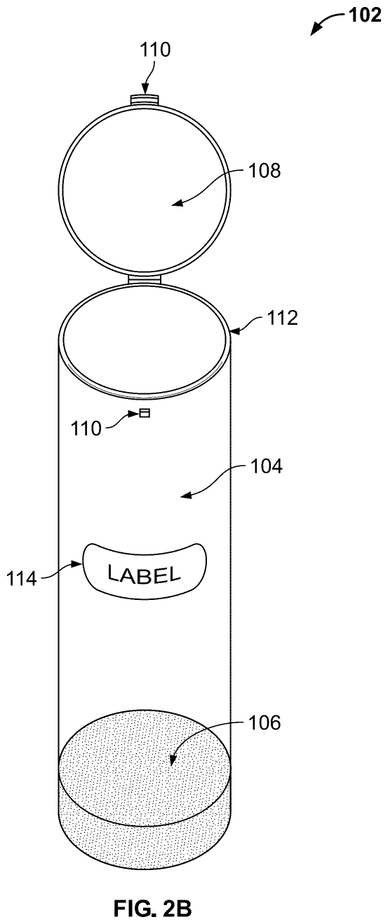

Referring now to FIGS. 2A and 2B, an example container 100 is described. The container 100 can include a plurality of compartments 102a, 102b, 102c, 102d, 102e (referred to herein individually and collectively as “compartment 102” and “compartments 102”). In FIG. 2A, the container 100 includes five compartments 102. It should be understood that the number of compartments shown in FIG. 2A is provided only as an example. This disclosure contemplates that the container 100 can include more or less than five compartments. Additionally, it should be understood that the shape and arrangement of the compartments 102 shown in FIG. 2A is provided only as an example. This disclosure contemplates the compartments of the container 100 can have a different shape and/or arrangement than shown in FIG. 2A.

As shown in FIG. 2B, each compartment 102 defines a respective internal volume 104. Additionally, the container 100 can include a plurality of odorous materials 106 (referred to herein individually and collectively as “odorous material 106” and “odorous materials 106”). Each compartment 102 houses a respective odorous material 106 within its respective internal volume 104. Each odorous material 106 can be, but is not limited to, a liquid, an aerosolized liquid, a solid, a polyurethane, a plastic, a powder, a gel, a nanoemulsion, a wax, a paper, a celluloid material, a gum base, a foam, or a composite. In some implementations, the odorous material 106 is provided directly on a substrate that is disposed in the internal volume 104. Optionally, the substrate can be a foam or a composite such as a polyurethane foam composite. For example, the odorous material 106 can optionally be provided on a polyurethane foam composite (e.g. a substrate). In other implementations, the odorous material 106 is absorbed into a secondary material, which is added to the internal volume 104. Additionally, each of the odorous materials 106 is configured to emit a distinct odor. Optionally, in some implementations, the distinct odor is a combination of a plurality of distinct odors. Distinct odors include, but are not limited to, a lemon odor, a vanilla odor, a floral odor, a peppermint odor, an apple odor, or a chocolate odor. It should be understood that lemon, vanilla, floral, peppermint, apple, and chocolate are provided only as example distinct odors. This disclosure contemplates that the distinct odors may be a different odor, including but not limited to, another pleasant odor.

Optionally, in some implementations, each compartment 102 can further include a respective cover 108 configured to restrict access to the respective internal volume 104. In some implementations, the cover 108 only restricts access to the internal volume 104 (i.e., it does not prevent leakage or contamination). In other implementations, the cover 108 is configured to prevent leakage of the odorous material 106 from the compartment 102 and/or to prevent contamination of odorous material 106 (e.g., mixing with foreign substances).

Optionally, in some implementations, each compartment 102 can further include a respective lock 110. For example, as shown in FIG. 2B, a portion of the lock 110 can be arranged on each of the cover 108 and the compartment 102. When the cover 108 is closed (i.e., restricting access to the internal volume 104), the lock 110 is engaged and configured to restrict the cover 108 from opening. On the other hand, when the cover 108 is open (i.e., as shown in FIG. 2B), the lock 110 is disengaged. This disclosure contemplates that the lock 110 can be mechanical, magnetic, electrical, or combinations thereof. It should be understood that the shape, size, and arrangement of the lock 110 shown in FIG. 2B is provided only as an example. This disclosure contemplates that the lock can have a different shape, size, and/or arrangement than shown in FIG. 2B.

Optionally, in some implementations, each compartment 102 can further include a respective seal 112. The seal 112 is configured to isolate the respective internal volume 104 from an external environment. Alternatively or additionally, the seal 112 is configured to isolate the respective internal volume 104 from the internal volumes of other compartments 102. In FIG. 2B, the seal 112 is arranged between respective mating surfaces of the cover 108 and the compartment 102. Thus, the seal 112 is configured to prevent leakage of the odorous material 106 from the compartment 102 and/or to prevent contamination of odorous material 106 (e.g., mixing with foreign substances). For example, the seal 112 can optionally be a gasket. A gasket is a mechanical seal that is used to fill the gap between mating surfaces, typically to prevent leakage from, or the ingress of foreign substances into, a system. A gasket can be made from flexible materials, such as rubber, silicone, cork, or various types of metal. It should be understood that a gasket is provided only as an example seal. Additionally, it should be understood that the shape, size, and arrangement of the seal 112 shown in FIG. 2B is provided only as an example. This disclosure contemplates that the seal can have a different shape, size, and/or arrangement than shown in FIG. 2B.

Optionally, in some implementations, each compartment 102 can further include a respective marking 114. The marking 114 indicates the odorous material 106 within the internal volume 104. For example, the marking 114 can indicate the type of odorous material 106, for example, lemon, vanilla, floral, peppermint, apple, or chocolate odor. In some implementations, the marking 114 is a visual marking. Visual markings include, but are not limited to, colors, numbers, words, lights (e.g. light-emitting diodes (LEDs)), or combinations thereof. Alternatively or additionally, the marking 114 is a tactile marking. Tactile markings include, but are not limited to, raised or textured features. In FIG. 2B, the marking 114 is on an external surface of the compartment 102. It should be understood that the shape, size, and arrangement of the marking 114 shown in FIG. 2B is provided only as an example. This disclosure contemplates that the marking can have a different shape, size, and/or arrangement than shown in FIG. 2B.

As noted above, the IC chip 150 can be programmed to perform one or more logical operations. For example, the IC chip 150 can be configured to monitor a respective state of each compartment 102. This disclosure contemplates that the container 100 can include one or more sensors (not shown) arranged in or on the compartment 102 and/or the cover 108. The IC chip 150 can be operably coupled with the sensor(s) and configured to receive signal(s) from such sensor(s) to determine a state of a compartment 102. Compartment states can include, but are not limited to, an opened state, a closed state, a locked state, or an unlocked state.

Optionally, in some implementations, the IC chip 150 can be configured to record the respective state of each compartment 102. This disclosure contemplates that the IC chip 150 can store the compartment state, for example, in its memory. Such information can be retrieved and/or accessed as needed. Optionally, in some implementations, the IC chip 150 can be configured to transmit the respective state of each compartment 102 to the remote computing device 180.

Optionally, in some implementations, the IC chip 150 can be configured to generate a control signal configured to prompt the subject to open a specific compartment 102 of the container 100. For example, the control signal may unlock one or more of the compartments 102, energize one or more LED indicators (e.g., markings 114), energize a piezoelectric actuator (e.g., vibration), or combinations thereof. Upon receipt of the prompt, the subject opens the cover 108 of the specific compartment 102 of the container 100. It should be understood that this exposes the subject to the odor contained in the specific compartment 102 of the container 100. Optionally, prompts facilitate providing selective access to one or more of the compartments 102.

As noted above, the remote computing device 180 can be programmed to perform one or more logical operations. Optionally, the logical operations are implemented by an application such as a mobile app or web-based application. The IC chip 150 and the remote computing device 180 can be operably coupled by a communication link as described above with regard to FIG. 1. Thus, the IC chip 150 and the remote computing device 180 can exchange information. In some implementations, the remote computing device 180 can be configured to receive a signal indicating the respective state (e.g., open/closed, locked/unlocked) of each compartment 102. Optionally, the remote computing device 180 can be further configured to monitor when compartment(s) 102 are opened/closed. Alternatively or additionally, the remote computing device 180 can be further configured to measure a respective amount of time each compartment 102 is in a particular state. For example, the remote computing device 180 can be configured to measure an amount of time a compartment 102 is open. It should be understood that monitoring the states of compartments can be used to understand if, when, and/or for how long the subject is exposed to the odors contained in the container 100.

Optionally, in some implementations, the remote computing device 180 can be further configured to: receive an image of the container 100; and analyze the image to determine the respective state of each compartment. It should be understood that monitoring the states of compartments using imaging can be used to understand if, when, and/or for how long the subject is exposed to the odors contained in the container 100.

Optionally, in some implementations, the remote computing device 180 can be further configured to prompt the subject to smell an odor contained in a specific compartment 102 of the container 100. Prompting the subject can include alerting the subject using a user interface (e.g., light, sound, vibration, etc.) of the remote computing device 180. Alternatively or additionally, prompting the subject can include, but is not limited to, transmitting a signal to the IC chip 150 to unlock one or more of the compartments 102, energize one or more LED indicators (e.g., markings 114), energize a piezoelectric actuator, or combinations thereof. Upon receipt of the prompt, the subject can open the cover 108 of the specific compartment 102 of the container 100. It should be understood that this exposes the subject to the odor contained in the specific compartment 102 of the container 100.

Optionally, in some implementations, the remote computing device 180 can be further configured to: receive, from a wearable sensor, a physiological signal associated with the subject; and analyze the physiological signal. The subject can be prompted to smell the odor contained in the specific compartment 102 of the container 100 based, at least in part, on the analysis. Physiological signals can include, but are not limited to, heart rate (HR) and heart rate variability (HRV). It should be understood that HR and HRV are only provided as examples. This disclosure contemplates receiving other physiological signals from a wearable sensor. Wearable sensors include, but are not limited to, a heart rate monitor, accelerometer, gyroscope, electrocardiogram (ECG/EKG) sensor, skin temperature sensor, blood oxygen (SpO2) sensor, and sweat sensor. Optionally, the wearable sensor is incorporated into remote computing device 180. Alternatively, the wearable sensor is optionally incorporated into another device such as a fitness tracker or smartwatch and transmitted to the remote computing device 180.

Optionally, in some implementations, the remote computing device 180 can be further configured to receive, from a location sensor, a location of the subject. A location sensor is used to determine the position or location of the subject. Global positioning system (GPS) and Wi-Fi position system (WPS) are example location sensors. It should be understood that GPS and WPS are only provided as examples. Optionally, the location sensor is incorporated into the remote computing device 180. Alternatively, the location sensor is optionally incorporated into another device such as the IC chip 150 or a smartwatch and transmitted to the remote computing device 180. The subject can be prompted to smell the odor contained in the specific compartment 102 of the container 100 based, at least in part, on the location of the subject. Alternatively or additionally, the subject can be prompted to smell the odor contained in the specific compartment 102 of the container 100 at a specific time. As used herein, the specific time may be a random, periodic, or predetermined time.

Optionally, in some implementations, the remote computing device 180 can be further configured to receive subject-reported information about the odor contained in the specific compartment 102 of the container 100. Such information may be feedback about the odor contained in the specific compartment 102 of the container 100 (e.g., whether such odor reduced a craving).

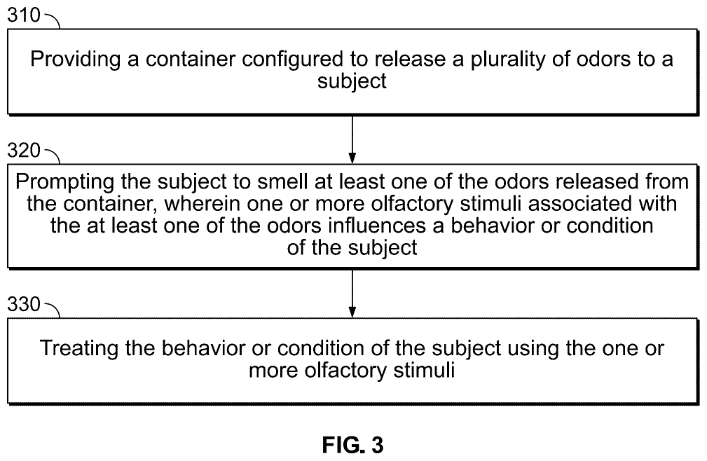

Referring now to FIG. 3, a flowchart illustrating example operations for using olfactory stimuli to influence a behavior or condition of a subject is described. As described above, in some implementations, the subject is a human. At step 310, a container configured to release a plurality of odors is provided to the subject. The container can be the container 100 described with regard to FIGS. 1-2B above. At step 320, the subject is prompted to smell at least one of the odors released from the container. One or more olfactory stimuli associated with the released odor(s) influences a behavior or condition of the subject. As described above, the released odor(s) may include a lemon odor, a vanilla odor, a floral odor, a peppermint odor, an apple odor, a chocolate odor, or combinations thereof. It should be understood that lemon, vanilla, floral, peppermint, apple, and chocolate are provided only as example odors. Additionally, as described herein, the behavior or condition can be smoking, alcohol consumption, drug use, food intake, stress, memory, an emotional state, or a compulsion. It should be understood that the above behaviors and conditions are provided only as examples. This disclosure contemplates that the behavior or condition may be another behavior or condition including, but not limited to, an unwanted behavioral or mental state. Optionally, at step 330, the behavior or condition of the subject is treated using the one or more olfactory stimuli.

It should be appreciated that the logical operations described herein with respect to the various figures may be implemented (1) as a sequence of computer implemented acts or program modules (i.e., software) running on a computing device (e.g., the computing device described in FIG. 4), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 4, an example computing device 400 upon which the methods described herein may be implemented is illustrated. It should be understood that the example computing device 400 is only one example of a suitable computing environment upon which the methods described herein may be implemented. Optionally, the computing device 400 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 400 typically includes at least one processing unit 406 and system memory 404. Depending on the exact configuration and type of computing device, system memory 404 may be volatile (such as random access memory (RAM)), non-volatile (such as read-only memory (ROM), flash memory, etc.), or some combination of the two. This most basic configuration is illustrated in FIG. 4 by dashed line 402. The processing unit 406 may be a standard programmable processor that performs arithmetic and logic operations necessary for operation of the computing device 400. The computing device 400 may also include a bus or other communication mechanism for communicating information among various components of the computing device 400.

Computing device 400 may have additional features/functionality. For example, computing device 400 may include additional storage such as removable storage 408 and non-removable storage 410 including, but not limited to, magnetic or optical disks or tapes. Computing device 400 may also contain network connection(s) 416 that allow the device to communicate with other devices. Computing device 400 may also have input device(s) 414 such as a keyboard, mouse, touch screen, etc. Output device(s) 412 such as a display, speakers, printer, etc. may also be included. The additional devices may be connected to the bus in order to facilitate communication of data among the components of the computing device 400. All these devices are well known in the art and need not be discussed at length here.

The processing unit 406 may be configured to execute program code encoded in tangible, computer-readable media. Tangible, computer-readable media refers to any media that is capable of providing data that causes the computing device 400 (i.e., a machine) to operate in a particular fashion. Various computer-readable media may be utilized to provide instructions to the processing unit 406 for execution. Example tangible, computer-readable media may include, but is not limited to, volatile media, non-volatile media, removable media and non-removable media implemented in any method or technology for storage of information such as computer readable instructions, data structures, program modules or other data. System memory 404, removable storage 408, and non-removable storage 410 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory 404. For example, the bus may carry data to the system memory 404, from which the processing unit 406 receives and executes instructions. The data received by the system memory 404 may optionally be stored on the removable storage 408 or the non-removable storage 410 before or after execution by the processing unit 406.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

In an example implementation, a multi-compartment device contains distinct odors (e.g., lemon, orange, chocolate) that can be accessed on demand (see FIG. 2A). For example, an individual may open compartment 102a to smell a lemon odor, compartment 102b to smell an orange odor, compartment 102c to smell a chocolate odor, compartment 102d to smell a floral odor, and compartment 102e to smell a peppermint odor. Optionally, the compartments 102 are color coded. Note that the color of the compartment does not need to be conceptually related to the odor. The various odor compartments can also be made distinct by tactile variations or by numbers or words. This multi-compartment device may be linked via wireless technology (e.g., Bluetooth) to a mobile device (e.g., smartphone, tablet) that prompts users to smell a specific odor (contained in a specific compartment) at a specific time. In this iteration, the mobile application may unlock a specific compartment within the odor containing device to enable the user to only smell that odor. In addition, the odor containing device may send a signal to the mobile application that the device was opened/used.

Optionally, the mobile application provides a behavioral intervention and/or prompts individuals to smell specific odor compartments as part of a multi-component intervention. Optionally, a wearable device (e.g., activity monitor) is used to collect physiological data in real time (e.g., heart rate, heart rate variability) and then prompt the user to smell a specific compartment of the odor device and confirm that the odor device was opened.

The odor may be sampled through a variety of physical devices that may have multiple odor compartments, is portable, and the odor samples are easily resealable or reseal themselves. To address these needs, the odors are most likely to be formulated in a solid, gum, or gel medium. The present disclosure merges a device containing odors in a format (e.g., gel) for delivering a menu of odors in a fashion that is convenient to use, easily portable and does not allow cross odor contamination or contaminate the user, that offers a selection of fragrances that affect cognition, emotion and behaviors.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A system for using olfactory stimuli to influence a behavior or condition of a subject comprising:
a container comprising:
a plurality of compartments, wherein each compartment defines a respective internal volume; and
a plurality of odorous materials, wherein each compartment houses a respective odorous material within the respective internal volume; and
an integrated circuit (IC) chip comprising a processor and a memory, the memory having computer-executable instructions stored thereon that, when executed by the processor, cause the IC chip to monitor a respective state of each compartment.

2. The system of claim 1, wherein the respective state of each compartment is at least one of an opened state, a closed state, a locked state, or an unlocked state.

3. The system of claim 1, wherein the memory has further computer-executable instructions stored thereon that, when executed by the processor, cause the IC chip to record the respective state of each compartment.

4. The system of claim 1, wherein the memory has further computer-executable instructions stored thereon that, when executed by the processor, cause the IC chip to transmit the respective state of each compartment to a remote computing device.

5. The system of claim 1, wherein the memory has further computer-executable instructions stored thereon that, when executed by the processor, cause the IC chip to generate a control signal configured to prompt the subject to open a specific compartment of the container.

6. The system of claim 1, wherein each odorous material comprises a liquid, an aerosolized liquid, a solid, a polyurethane, a plastic, a powder, a gel, a nanoemulsion, a wax, a paper, a celluloid material, a gum base, a foam, or a composite.

7. The system of claim 1, wherein each of the odorous materials is configured to emit a distinct odor.

8. The system of claim 7, wherein the distinct odor is a lemon odor, a vanilla odor, a floral odor, a peppermint odor, an apple odor, or a chocolate odor.

9. The system of claim 1, wherein each compartment further comprises a respective cover configured to restrict access to the respective internal volume.

10. The system of claim 9, wherein each compartment further comprises a respective lock.

11. The system of claim 1, wherein each compartment further comprises a respective seal configured to isolate the respective internal volume from an external environment.

12. The system of claim 1, wherein each compartment further comprises a respective marking to indicate the respective odorous material within the respective internal volume.

13. The system of claim 1, further comprising a remote computing device.

14. The system of claim 13, wherein the IC chip and the remote computing device are operably coupled by a communication link.

15. The system of claim 14, wherein the communication link is a wireless communication link.

16. The system of claim 13, wherein the remote computing device is configured to receive a signal indicating the respective state of each compartment.

17. The system of claim 16, wherein the remote computing device is configured to measure a respective amount of time each compartment is in an opened state.

18. The system of claim 13, wherein the remote computing device is configured to:
receive an image of the container; and
analyze the image to determine the respective state of each compartment.

19. The system of claim 13, wherein the remote computing device is configured to prompt the subject to smell an odor contained in a specific compartment of the container.

20. The system of claim 19, wherein the remote computing device is configured to receive subject-reported information about the odor contained in the specific compartment of the container.

21. The system of claim 19, wherein the remote computing device is configured to:
receive, from a wearable sensor, a physiological signal associated with the subject; and
analyze the physiological signal, wherein the subject is prompted to smell the odor contained in the specific compartment of the container based, at least in part, on the analysis.

22. The system of claim 19, wherein the remote computing device is configured to receive, from a location sensor, a location of the subject, wherein the subject is prompted to smell the odor contained in the specific compartment of the container based, at least in part, on the location of the subject.

23. The system of claim 19, wherein the subject is prompted to smell the odor contained in the specific compartment of the container at a specific time.

24. The system of claim 1, wherein the container is portable.

25-37. (canceled)
